(19) United States
(12) Patent Application Publication
(10) Pub. No.: US 2017/0095655 A1
(43) Pub. Date: Apr. 6, 2017

(54) Sterile Caps For IV Line Care

(71) Applicant: Jonathan D. Whitfield, Anderson, SC (US)

(72) Inventor: Jonathan D. Whitfield, Anderson, SC (US)

(21) Appl. No.: 15/281,890

(22) Filed: Sep. 30, 2016

Related U.S. Application Data
(60) Provisional application No. 62/235,749, filed on Oct. 1, 2015.

(51) Int. Cl. A61M 39/20

(52) U.S. Cl. CPC A61M 39/20; A61M 2039/0036

(57) ABSTRACT

This invention is directed to a sterile IV tube end cap comprising: a housing attached to a strip; an end cap received in the housing and removably attached to the strip; so that when the IV tube is attached to the end cap, the IV tube cannot be removed from the end cap without removing the end cap from the strip. The end cap can receive the male end of an IV tube wherein a portion of the IV tube is received in the housing and the IV tube is secured to the strip so that the IV tube cannot be removed from the end cap without removing the end cap from the strip.

### BACKGROUND OF THE INVENTION

1) Field of the Invention

The present invention relates to a sterile IV tube end cap that is used to prevent contamination of the IV tube and improve nurse compliance with IV use procedures.

2) Description of Related Art

In the medical field, there is an area coined intravenous (“IV”) therapy. IV therapy can be administered through both peripheral and central line sites and are of great interest to the healthcare industry. Typically, the IV needle is inserted into the patient. The IV needle can include a female luer connection. The IV tube can include a male connection that is connected to the IV needle on one end and fluid source or other object at the other end. One challenge with IV therapy lies with potential infections related to peripheral and central IV therapy medical devices that can promote problems at injection sites, or within the lines of medication administration. Such complications are directly reflected in hospital grading and insurance reimbursement, specifically, the central line associated blood stream infections (CLABSIs) and instances of sepsis. This problem can occur when the male end of the IV tube comes in contact with a non-sterile surface, fluid or solid and is then attached to the needle.

Attempts to reduce these complications include devices and care procedures that have been created to help prevent the contamination of IV lines. For example, procedures have been put in place where nurses use individually packaged swabs of alcohol to scrub female IV port (or “hub”) connectors to disinfect them prior to connecting a male fitting syringe or male luer lock fitting in order to infuse medication through an IV site. Such attempts at covering or cleaning the connectors include U.S. Pat. No. 8,419,713 directed to an assembly for mounting medical connector caps includes a carrier formed from a sheet of material; US Application Publication 2012/0216359 directed to cleaning system for medical implements includes a number of cleaning caps attached to a substrate; U.S. Pat. No. 8,834,650 direct to a system for cleaning a site of a medical implement; and U.S. Pat. No. 8,641,681 directed to disinfecting caps that can be used to cover and disinfect a male luer post of a medical connector. However, none of these attempts provide an assembly for securely holding the connector and preventing contamination of the connector in the first place.

Devices such as sterile luer lock caps are used to protect both male and female ends of luer lock IV sets. Some caps (like the CUROS® cap) have instilled a form of disinfecting agent, such as isopropyl alcohol, within both male and female caps to provide further assurance that these surfaces are free from germs; however, these attempts to provide a solution do not correctly identify or remedy the actual source of the problem. Assisting nurses to comply with IV capping procedures is the task at hand, as prior devices are not beneficial if nurses do not consistently use them, or use them improperly. Without compliance to procedures designed to keep the open male and/or female ends of IV sets free from contaminants, previous devices have not served any purpose at all.

In practice, nurses can fail to follow procedures by: simply not applying sterile caps to the open ends of IV lines (primary and/or secondary); “looping” the open male end of the line back and connecting it into a female port or end of itself, to create a circle that makes it much easier to manage by then being able to hang from an IV pole or other hanger device; capping the end of the line correctly, only to have it fall to the floor in an attempt to somehow balance the line over the IV pump itself or within the hooks of an IV pole or other hanger to make the line more manageable and secure from falls. Nurses must choose to replace dropped lines (safe practice), or continue using them with possible contamination. Even with a capped line, allowing it to come into contact with the floor increases the risk for infection to enter the blood stream through the IV site.

Additionally, balancing the end of the line somewhere over a pump or IV stand also makes the reuse of the line an inconvenience as one tries to find the end of the line and avoid having it come in contact with the floor, at which point the entire IV line would need replacement. The above did not mention the time it takes to replace entire IV sets, nor the time wasted untangling multiple lines hanging from IV poles. Sorting and managing multiple IV medications with dangling lines can be a nuisance for nurses dealing with complex patients being treated with multiple IV drugs or solutions, and can even potentiate medication administration mistakes.

First, the numerous individual caps placed over male connectors each instance the line is disconnected is time consuming to unpackage, and leaves nurses with more waste to place in receptacles, which also becomes a roadblock to compliance for the end-user. Caps that are attached to a “strip” hanging from IV poles that can be easily peeled off and applied are much more user friendly, and less time consuming to use, which can help increase nurse “correct capping compliance.” These detachable male caps produce less waste and are more readily available; however they still do not solve the glaring problem of nurse compliance and complete ease and willingness of use. Individual, loose cap pieces (even when peeled from a hanging strip) still leaves the nurse hunting for a place to balance the male ends of IV lines over pole hooks or IV pumps, etc., to make them easily accessible each time the line is needed, and leaves many lines dangling to the hospital floor, a breeding ground for infection. And the caps, once removed, are many times placed on the tops of IV pumps or on a nearby surface and reused when the line is again unhooked from the patient.

There is a need for a solution that reduces the tangled IV lines, prevents sterile lines from falling to the floor, helps insure that nurses will only use a cap once per disconnect and reduce the need for a nurse to “loop” an IV line back into itself to create a hanger to make a line more accessible.

Therefore, it is an object of the present invention to provide a medical device directed for simplifying the process for medical professionals to follow the correct procedure when securing and capping the male end of an IV line.

It is another object of the present invention to provide an assembly that secures the connector to reduce or prevent contamination.

It is another object of the present invention to provide a “one time use” assembly to further reduce or prevent contamination.

### SUMMARY OF THE INVENTION

The above objectives are accomplished according to the present invention by providing a sterile IV tube end cap comprising: a housing attached to a strip; an end cap received in the housing and removably attached to the strip; a housing sloped portion defined on an inner wall of the housing; an end cap sloped portion cooperatively associated with the housing sloped portion so that when the IV tube is attached to the end cap, the IV tube cannot be removed from the end cap without removing the end cap from the strip; a first fastening member for removably attaching the end cap to the strip; and, whereas the end cap receives the male end of an IV tube wherein a portion of the IV tube is received in the housing and the IV tube is secured to the strip so that the IV tube cannot be removed from the end cap without removing the end cap from the strip. The invention can include a second fastening member for receiving the first fastening member and securing the housing to the strip.

The invention can include a one-way fastener in the strip for removably attaching the end cap to the strip. A resilient member and a rigid member can be included in the one-way fastener. In one embodiment the resilient member can be generally conical in shape.

The invention can include a housing affixed to a strip; a hook opening defined in the strip configured to allow the strip hand from a hook such as on an IV pole; a discontinuous area configured to allow multiple strips to be connected to each other forming a chain of strips; an open end defined in the housing; an end cap received in the open end of the housing and releasably attached to the strip; a housing sloped portion defined on an inner wall of the housing; an end cap sloped portion cooperatively associated with the housing sloped portion so that when a proximal end of an IV tube is attached to the end cap, the proximal end cannot be removed from the end cap without removing the end cap from the strip; a resilient member carried by the strip configured to receive a fastening member included in the end cap; a tab removably attached to the open end of the housing configured to cover the housing prior to the proximal end of the IV tube being received by the housing; a sterilization pad included in a control opening defined in the end cap carrying a sterilization agent; and, a circumferential flange included in the end cap configured to engage with the proximal end of the IV tube securing the end cap to the proximal end.

The resilient member can include a rigid ring and a resilient ring. The fastening member can extend through the strip. A portion of the fastening member that extends through the strip can be received into a second fastening member. The fastening member can be a ball and socket arrangement. The control opening can receive a male portion of the proximal end of the IV tube. The end cap can be configured to rotate within the housing.

### BRIEF DESCRIPTION OF THE DRAWINGS

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1 is a view of an IV line as is known in the art;

FIG. 2 is a perspective view of aspects of the invention;

FIG. 3 is an end view of aspects of the invention;

FIG. 4 is a perspective view of aspects of the invention;

FIG. 5 is a perspective view of aspects of the invention;

FIG. 6 is an elevation view of aspects of the invention; and,

FIG. 7 is a perspective view of aspects of the invention.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### DETAILED DESCRIPTION OF A PREFERRED EMBODIMENT

With reference to the drawings, the invention will now be described in more detail. Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are herein described.

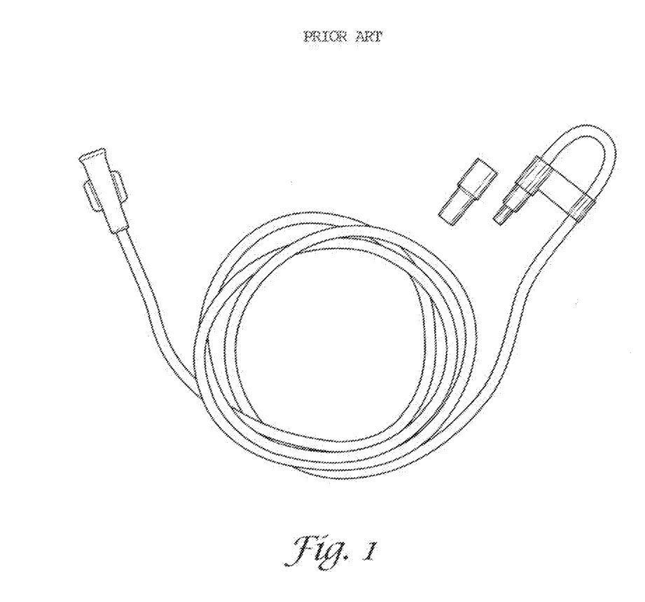

Referring to FIG. 1, an IV line 10 can include a distal end 12 that includes a male end 14. The male end can be a male luer end. The distal end can be attached to a female end of an IV catheter extension that then would connect to a needle and cannula that is inserted into the patient with a portion of the distal end of the tubing possibly taped to the patient near the insertion site. A distal (male) end cap 16 can be used to cap the distal end of the IV tube. A proximal end 18 can be included in the IV tube and can include a female end 20. An end cap 22 can be attached to the proximal (female) end. In use, the proximal end is connected to any number of articles including IV medication through the use of other IV connectors. Other female ends, or ports such as the female end 20 can extend from any portion of the tubing 10 to create an access point for medication administration through the IV line. It is the distal end 14 that is normally subject to reduced sterility when it is disconnected from the patient without being capped, capped incorrectly, falls to the floor, or with other exposure and the like. When the IV is attached to the end cap and the end cap is attached to the strip, the IV tube can hang from the end cap and therefore does not contact the floor. This feature can prevent the IV tube from being contaminated as well as prevent tangling of the tubes themselves.

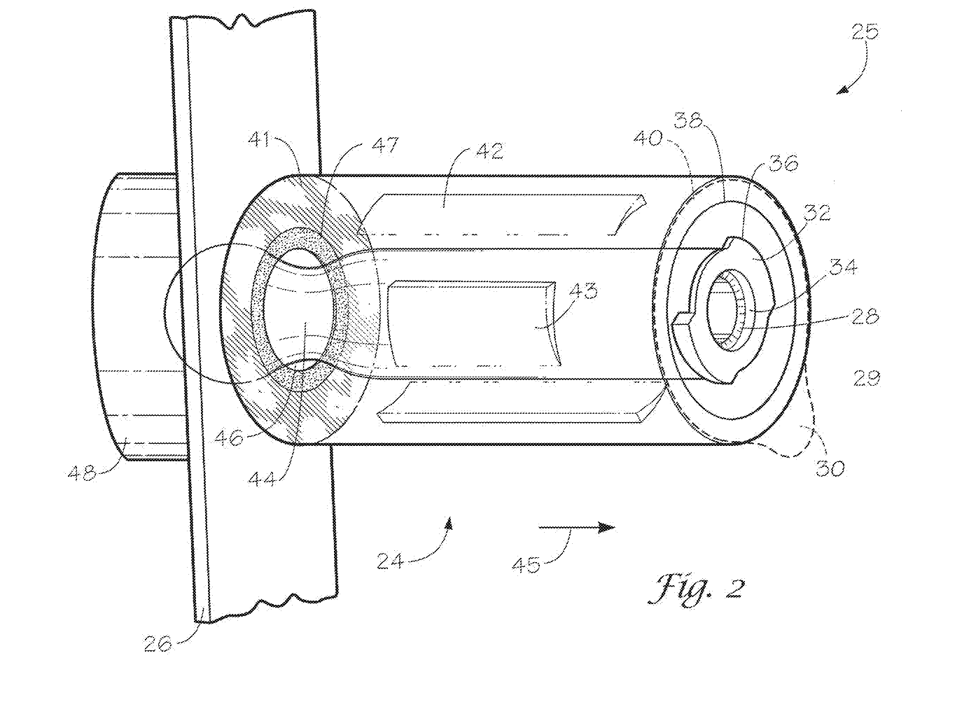

Referring to FIG. 2, an assembly 24 is attached to a strip 26. In one embodiment, the strip hangs from the IV stand for quicker availability for nurse. The strip allows an end cap 32 to be readily accessible to the nurse for use. The open end 25 of the cap can have a tear away cover 28 with a tab 30. By providing the tear away cover, the open end of the end cap is covered and sealed to help insure sterility until the nurse is ready to use the cap. The assembly can include an end cap 32 with a central opening 34 and circumferential flanges 36 that can completely or partially surround the central opening. The central opening can include a sterilization pad or swab 29 attached to the interior of the central opening so that when the end of the IV tube is received in the central opening, the end of the IV tube is sterilized with a sterilization agent.

A swivel portion of an end of an IV tube can be received into the opening defined between the housing 38 and the end cap. In one embodiment, the swivel portion of the male end is received into the housing. The male portion of a catheter would be received into the center 34 of the end cap. The inner wall 40 of the housing can include a housing sloped portion 42 that cooperates with an end cap sloped portion 43 disposed on the outer surface of the end cap so that the IV tube cannot be removed from the end cap without the end cap being removed from the strip. The IV connector can be disposed so that when attached to the end cap, the swivel portion does not extend into the area occupied by the housing sloped portion 42 in one embodiment. The housing sloped portion and end cap sloped portion can be cooperatively associated to prevent the IV tube from being removed from the end cap unless the end cap is removed from the strip.

The end cap can include a first fastening member 44 that can be received into the opening 46 in the strip. In one embodiment the diameter of the first fastening member is larger than the opening so that sufficient force is required to deform the opening to cause the end cap to be removed from the strip. The opening can be defined in the strip or in a second fastening member 48. The first fastening member can extend through the strip and be received into the second fastening member so that the end cap is secured to the strip. In one embodiment, once the end cap is removed from the second fastening member, the second fastening member cannot be replaced preventing reuse of the end cap and promoting compliance with proper IV procedures. In one embodiment, the first fastening member and second fastening member are a ball and socket assembly. In one embodiment, when the end cap is removed from the strip, the housing remains attached to the strip. In one embodiment, the housing is attached to the strip and surrounds the end cap so that when the IV tube is attached to the end cap, the IV tube cannot be separated from the end cap without the end cap being removed from inside the housing. In one embodiment, the housing cannot be removed from the strip. In one embodiment, the second fastening member is not attached to the strip and held in place when the end cap is in place. The second fastening member drops away when the end cap is removed.

In one embodiment, a one-way fastener is included in the strip allowing the end cap to be removeably attached to the strip, but not to be replaced on the strip. A rigid ring 41 can define the opening in the strip for receiving the first fastening member. A resilient ring 47 is disposed within the housing and adjacent to the end cap when the end cap is attached to the strip. When the end cap is pulled away from the strip in a direction shown as 45, the resilient member deforms inward allowing the first fastening member to be pulled through the opening and removed from the strip. When the end cap is attempted to be replaced by pushing it in a direction opposite that of 45, the resilient member contacts the rigid member and does not sufficiently deform to allow the first fastening member to be placed through the opening in the strip, thereby preventing the end cap from being reused. The rigid portion 41 can be disposed away from end 25 in relation to the resilient member 47 in one embodiment.

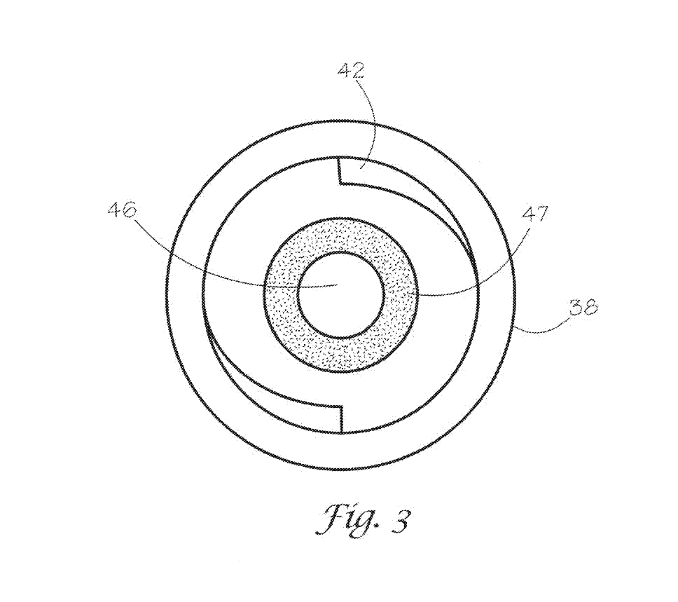

Referring to FIG. 3, the open end of the housing is shown. The opening as shown allows the resilient member 47 to be exposed after the end cap has been removed. The portion shown as 47, in one embodiment, is the portion of the resilient member that can deform when the end cap is pulled from the strip.

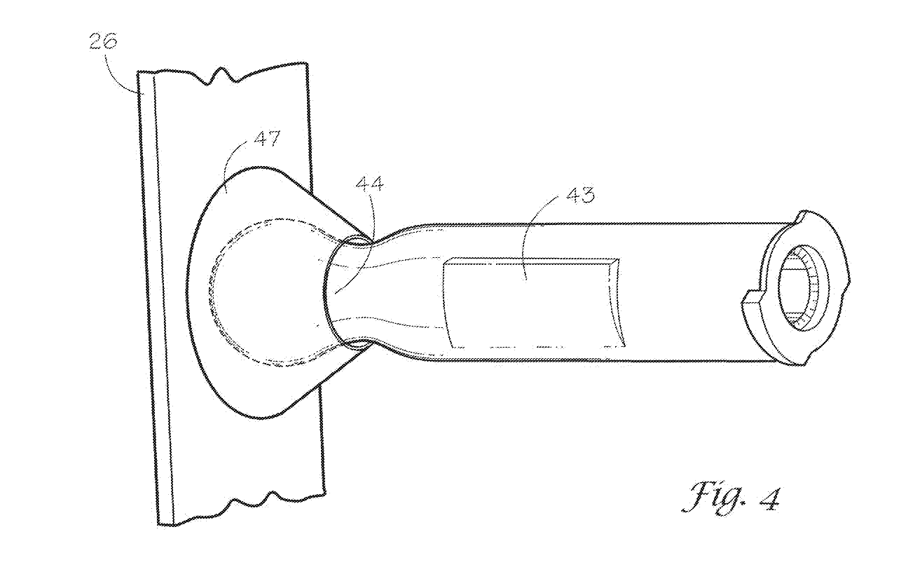

Referring to FIG. 4, the end cap is shown without the housing. The end cap is attached by a first fastening member 44 to a resilient member 47. In this embodiment, the resilient member is attached to the strip 26 so that the first fastening member does not extend through the strip. When the end cap is pulled from the resilient member, the resilient member deforms so that the end cap can be removed. The resilient member reverts generally to its original shape preventing the end cap from being reattached to the strip to deter reuse of the end cap and increase compliance with IV procedures. In one embodiment, the resilient strip is generally a conical shape.

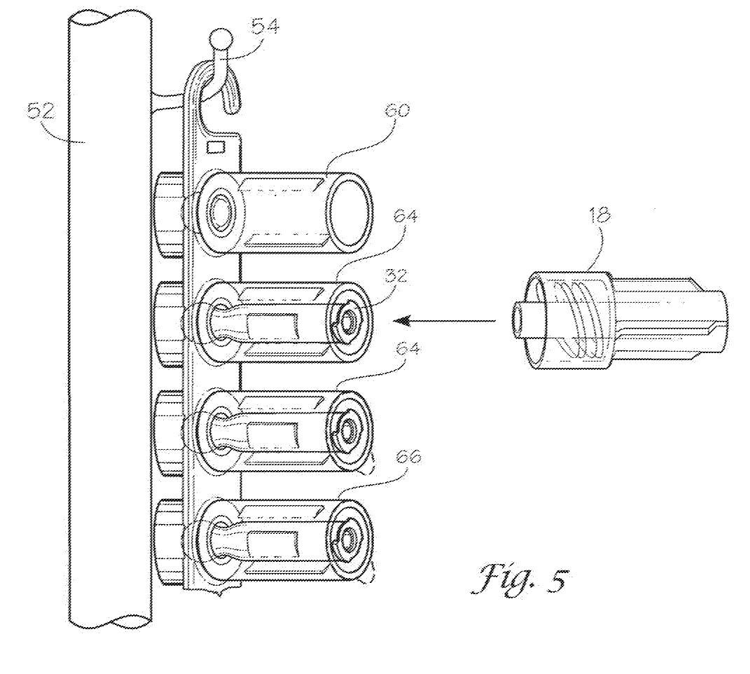

Referring to FIG. 5, the end caps can be positioned on the strip and can hang from the IV stand 52 from a hook 54 or another hanging element. The top housing 60 is shown after the end cap has been removed. The next housing 62 is shown with the seal removed and a IV end 18 being placed on the end cap. The next housings 64 and 66 are shown with the seals in place and ready for use.

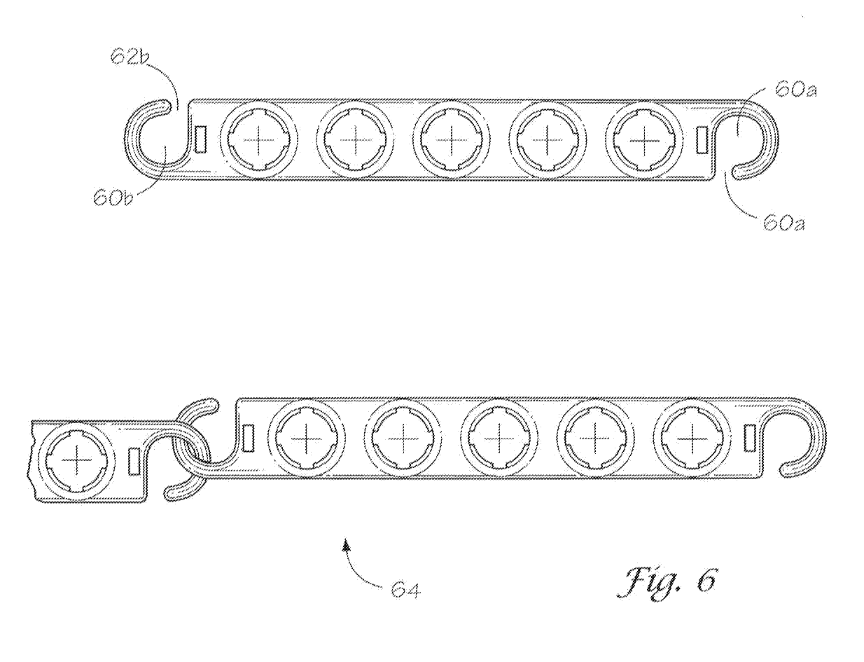

Referring to FIG. 6, in one embodiment, the strip can include opening 60a and 60b at each end that include discontinuous area 62a and 62b. the discontinuous area allowing adjacent strips to be connected to each other in a chain 64.

This invention results in a higher rate of compliance among healthcare providers in charge of intravenous line care for both central and peripheral lines. Having a higher rate of compliance among providers translates directly to a lower rate of infection, improved patient outcomes, and lower healthcare costs for facilities providing IV care. The end cap can be firmly attached to the strip and cannot be removed without sufficient effort.

By offering an end cap attached to a strip that is hung from IV poles, nurses are provided with a safer, easier, sterile alternative to the method of “looping” the IV lines into themselves (found very often among nurses) while offering more ease of use and ready availability of the IV line for later use. This also allows nurses to more easily organize and identify multiple IV lines, keeping drugs for more complex patients in order, which will minimize time consumption and mistakes. Not only would hanging stationary IV caps in this manner make it much easier for nurses to comply with hospital regulations, but it would also make them much more willing to do so, which is key in solving the problem of end-user compliance.

The end cap in one embodiment must be detached from its “base” in order for a nurse to uncap the male portion of the IV line. The actual cap portion only covers the central, thin, male catheter portion of the line, and is attached to the cap base using a ball-in-socket type of joint. The cap is unable to be removed by unscrewing it, but must be unsnapped from its base rendering it more difficult to be reused as opposed to using a new cap from the cap strip. This functionality increases compliance with IV procedures. Also, for the instances where a nurse needs a capped line away from the IV pole, it is ready and available by simply snapping the cap off of its base.

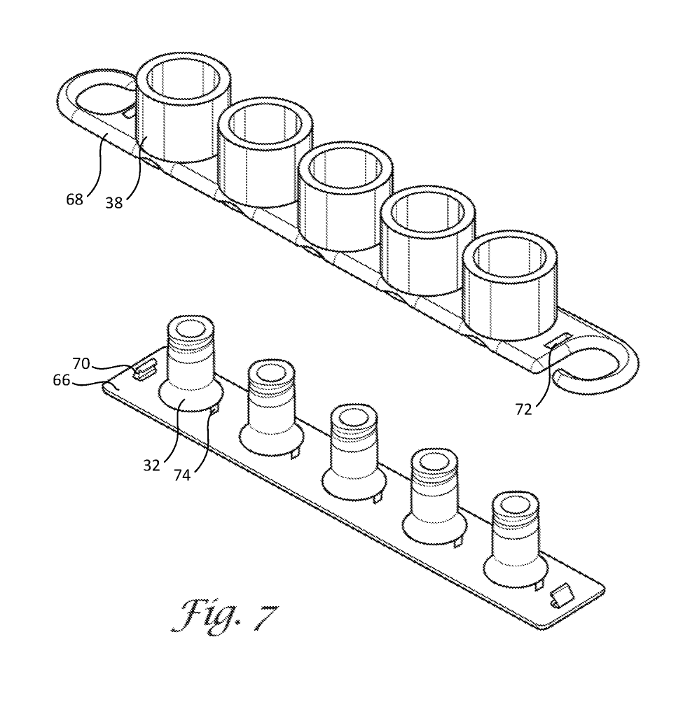

Referring to FIG. 7, the strip can include a rear portion 66 that can include a tab 70. The rear portion can be attached to the front portion 68 and affixed to the front portion when the tab is received into a tab opening 72. The housing 38 can be attached to the front portion. The end cap 32 can be attached to the rear portion. In one embodiment, the end cap is attached to the rear portion by a breakaway connection 74.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

While the present subject matter has been described in detail with respect to specific exemplary embodiments and methods thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art using the teachings disclosed herein.

What is claimed is:

1. An IV tube end cap and support assembly comprising:
a housing affixed to a front portion of a strip;
a hook opening defined in the strip configured to allow the strip to hang from a hook;
an opening defined in the housing;
an end cap received in the opening of the housing and releasably attached to a second portion of the strip;
a housing sloped portion defined on an inner wall of the housing;
an end cap sloped portion cooperatively associated with the housing sloped portion so that when a proximal end of an IV tube is attached to the end cap, the proximal end cannot be removed from the end cap without removing the end cap from the strip;
a tab removably attached to an open end of the housing configured to cover the open end of the housing prior to the proximal end of the IV tube being received by the housing;
a sterilization pad included in a control opening defined in the end cap carrying a sterilization agent; and,
a circumferential flange included in the end cap configured to engage with the proximal end of the IV tube securing the end cap to the proximal end.

2. The assembly of claim 1 wherein the first portion and the second portion of the strip are configured to attached to each other to define the strip.

3. The assembly of claim 1 wherein the end cap extends through the strip.

4. The assembly of claim 3 wherein the end cap is configured to be one-time releasably attached to the strip.

5. The assembly of claim 1 including:
a ball attached to the end cap; and,
a socket carried by the strip wherein the ball is releasably attached to the strip.

6. The assembly of claim 1 wherein the control opening is configured to receives a male portion of the proximal end of the IV tube.

7. The assembly of claim 1 wherein the end cap is configured to rotate within the housing.

8. An IV tube end cap and support assembly comprising:
a strip;
a housing affixed to the strip;
an end cap received in each housing and releasably attached to the strip;
a housing sloped portion defined on an inner wall of the housing;
an end cap sloped portion defined in an outer wall of the end cap; and,
wherein the housing sloped portion and the end cap sloped portion are cooperatively associated so that a proximal end of an IV tube received by the end cap and housing cannot be removed from the housing without removing the end cap from the strip.

9. The assembly of claim 8 including a tab removably attached to an open end of the housing configured to cover the open end of the housing prior to the proximal end of the IV tube being received by the housing.

10. The assembly of claim 8 including a breakaway connection attaching the end cap to the strip.

11. The assembly of claim 8 including a circumferential flange included in the end cap configured to engage with a proximal end of the IV tube securing the end cap to the proximal end.

12. The assembly of claim 8 including a resilient member carried by the strip configured to receive a fastening member included in the end cap wherein the resilient member includes a rigid ring and a resilient ring.

13. The assembly of claim 12 wherein the fastening member extends through the strip.

14. The assembly of claim 13 wherein a portion of the fastening member extending through the strip is received into a second fastening member.

15. The assembly of claim 8 wherein the control opening receives a male portion of the proximal end of the IV tube.

16. An IV tube end cap and support assembly comprising:
a strip;
a housing affixed to the strip; and,
an end cap received in each housing and configured to be one-time releasably attached to the strip;

17. The assembly of claim 16 including:
a housing sloped portion defined on an inner wall of the housing;
an end cap sloped portion defined in an outer wall of the end cap; and,
wherein the housing sloped portion and the end cap sloped portion are cooperatively associated so that a proximal end of an IV tube received by the end cap and housing cannot be removed from the housing without removing the end cap from the strip.

18. The assembly of claim 16 including a sterilization pad included in a control opening defined in the end cap.

19. The assembly of claim 16 wherein a resilient member is attached to the strip and includes a rigid ring and a resilient ring configured to receive a portion of the end cap.

20. The assembly of claim 16 wherein a portion of the end cap extends through the strip.
